(19) United States
(12) Patent Application Publication
(10) Pub. No.: US 2022/0323345 A1
(43) Pub. Date: Oct. 13, 2022

(54) HUMANIZED ANTIBODY FOR SARS-CoV-2 SPIKE PROTEIN INFUSION

(71) Applicants: Arizona Board of Regents on behalf of Arizona State University, Scottsdale, AZ (US); Halberd Corporation, Jackson Center, PA (US)

(72) Inventors: Qiang Chen, Chandler, AZ (US); Mitchell Steven Felder, Hermitage, PA (US)

(21) Appl. No.: 17/717,703

(22) Filed: Apr. 11, 2022

Related U.S. Application Data
(60) Provisional application No. 63/173,763, filed on Apr. 12, 2021.

(51) Int. Cl. A61K 9/00; C07K 16/10; A61K 49/00; A61M 1/14

(52) U.S. Cl. CPC A61K 9/0019; C07K 16/10; A61K 49/0058; A61K 49/0041; A61K 49/0034; A61M 1/14; A61M 2202/206

(57) ABSTRACT

An embodiment provides a method for treatment of the COVID-19 virus, including: preparing an intravenous infusion, wherein the intravenous infusion comprises at least one humanized monoclonal antibody against the SARS-CoV-2 spike protein; introducing the intravenous infusion to a patient; forming a virion antibody complex, wherein the virion antibody comprises the at least one humanized monoclonal antibody against the SARS-CoV-2 spike protein; and monitoring the virion antibody complex from a sample of a body fluid. Other aspects are described and claimed.

### CROSS REFERENCE TO RELATED APPLICATION

This application claims priority to U.S. Provisional Patent Application Ser. No. 63/173,763, filed on Apr. 12, 2021, and entitled “HUMANIZED ANTIBODY FOR SARS-CoV-2 SPIKE PROTEIN INFUSION,” the contents of which are incorporated by reference herein.

### FIELD

This application relates generally to the treatment of Covid-19, and, more particularly, by utilizing humanized antibodies against the SARS-CoV2 Spike Protein in an intravenous infusion.

### BACKGROUND

Coronaviruses represent a group of viruses that may lead to respiratory tract infections. These infections may range from mild to lethal. Coronaviruses may cause severe acute respiratory syndrome (SARS) and Middle East respiratory syndrome (MERS). A novel coronavirus (COVID-19) has led to a global pandemic causing a public health and economic crisis. Severe acute respiratory syndrome coronavirus 2 (SARS-CoV-2) is the strain of coronavirus that causes coronavirus disease 2019. Transmission may be through close contact of individuals and via respiratory droplets such as coughs or sneezes. Faster and more accurate methods of identifying and treating individuals infected with COVID-19 could mitigate the global pandemic.

### BRIEF SUMMARY

In summary, one embodiment provides a method for treatment of the COVID-19 virus, comprising: preparing an intravenous infusion, wherein the intravenous infusion comprises at least one humanized monoclonal antibody against the SARS-CoV-2 spike protein; introducing the intravenous infusion to a patient; forming a virion antibody complex, wherein the virion antibody comprises the at least one humanized monoclonal antibody against the SARS-CoV-2 spike protein; and monitoring the virion antibody complex from a sample of a body fluid.

Another embodiment provides a treatment kit for treatment of the COVID-19 virus, comprising: prepare an intravenous infusion, wherein the intravenous infusion comprises at least one humanized monoclonal antibody against the SARS-CoV-2 spike protein; introduce the intravenous infusion to a patient; form a virion antibody complex, wherein the virion antibody comprises the at least one humanized monoclonal antibody against the SARS-CoV-2 spike protein; and monitor the virion antibody complex from a sample of the body fluid.

A further embodiment provides a method for treatment of the COVID-19 virus, comprising: preparing an intravenous infusion, wherein the intravenous infusion comprises at least one humanized monoclonal antibody against the SARS-CoV-2 spike protein; introducing the intravenous infusion to a patient; forming a virion antibody complex, wherein the virion antibody comprises the at least one humanized monoclonal antibody against the SARS-CoV-2 spike protein; monitoring the virion antibody complex from a sample of a body fluid; and removing the virion antibody complex from the body fluid.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example method for utilizing humanized antibodies against the SARS-CoV2 spike protein in an intravenous infusion.

### DETAILED DESCRIPTION

It will be readily understood that the components of the embodiments, as generally described and illustrated in the FIGURES herein, may be arranged and designed in a wide variety of different configurations in addition to the described example embodiments. Thus, the following more detailed description of the example embodiments, as represented in the FIGURES, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

COVID-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for COVID-19. What is needed is a treatment of COVID-19 in a patient.

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is known as the severe acute respiratory syndrome coronavirus 2 (SARS-CoV-2). The disease it causes is called coronavirus disease 2019 (COVID-19).

In a phylogenetic analysis of 103 strains of SARS-CoV-2 from China, two different types of SARS-CoV-2 were identified, designated type L (accounting for 70 percent of the strains) and type S (accounting for 30 percent). The strains in L type, derived from S type, are evolutionarily more aggressive and contagious.

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient. There is therefore a need for treatments to reduce Covid-19 symptomatology in a clinical setting.

Accordingly, an embodiment provides a method for treating COVID-19 in a patient. In an embodiment, an intravenous infusion, wherein the intravenous infusion comprises at least one humanized monoclonal antibody against the SARS-CoV-2 spike protein may be prepared. The intravenous infusion may be introduced into a patient's body. In an embodiment, body fluid may be exposed to at least one binding antibody. In an embodiment, a treatment may be applied to the body fluid. In an embodiment, a treatment may comprise exposing the body fluid to a binding antibody. The binding is to an antigen specific to the spike protein of SARS-CoV-2. In an embodiment, the method may determine the presence or absence of the virion binding antibody complex. In an embodiment, a characteristic of the patient may be monitored. The method may prevent the COVID-19 virus or mutations thereof from infecting further cells of the patient's body. The treatment may improve outcomes, decrease severity, shorten time to recovery, or the like.

The illustrated example embodiments will be best understood by reference to the FIGURES. The following description is intended only by way of example, and simply illustrates certain example embodiments.

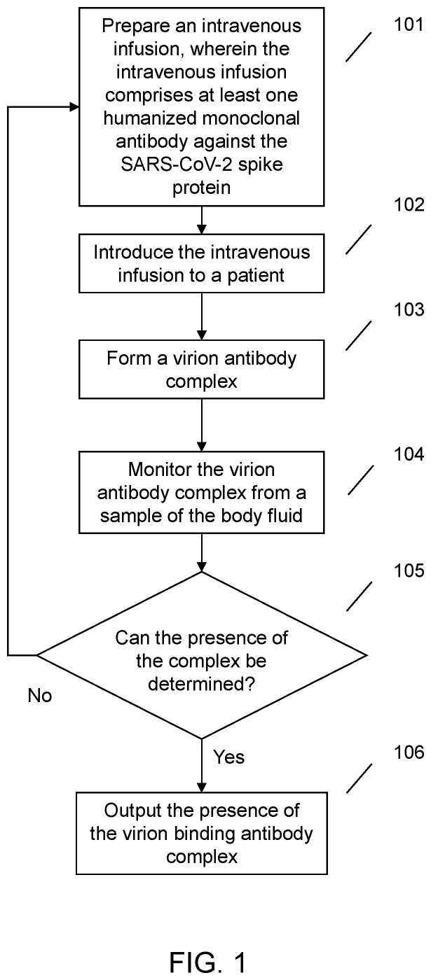

Referring to FIG. 1, an example device, kit, and method for preventing the COVID virus from infecting further cells is shown. The method may use one or more monoclonal antibodies. The antibody may be directed to the spike protein of SARS-CoV-2. The antibody may be directed to a portion of the SARS-CoV-2 protein. The antibody may comprise a fluorescent tag. In an embodiment, the body fluid may be exposed to at least one binding antibody, form a virion antibody complex. The exposure may be a form of an intravenous delivery system, infusion, or the like. The system and method may determine the presence of the virion binding antibody complex. It should be understood that the method and system described herein may be used for treatment and/or diagnostic purposes. In other words, the method may be used as a test kit for example at a medical facility, a testing facility, at home, or the like.

At 101, in an embodiment, an intravenous infusion or solution may be prepared. In an embodiment, the intravenous infusion may comprise at least one humanized monoclonal antibody. The humanized monoclonal antibody may be specific against the SARS-CoV-2 spike protein or another region of the SARS-CoV-2 protein. In an embodiment, a treatment for another species may be adapted and is contemplated and disclosed.

At 102, in an embodiment, the intravenous infusion may be performed on a patient. The patient may be diagnosed with, exhibit symptoms, or the like of infection with COVID-19 or a mutation thereof. Intravenous infusion may be performed using standard medical techniques. The intravenous infusion may expose the antibodies contained therein to a patient's body fluid. The body fluid may be blood, CSF (cerebrospinal fluid), mucus, saliva, or any bodily fluid. The body fluid may be from a patient which may contain COVID-19 virions. A virion may be a complete ineffective form of a virus outside of a host cell. A virion may be able to bind to a monoclonal antibody selective for the virion component. The component may include, but is not limited to genetic material such as DNA or RNA, protein, or the like. In an embodiment, the body fluid may be exposed to at least one binding antibody. For example, the antibody may be a monoclonal antibody and may selectively bind the spike protein of SARS-CoV-2. At 103, in an embodiment, the COVID-19 virions present in the body fluid may form a virion antibody complex with the binding antibody. The virion antibody complex is depicted at 103 in the flow diagram.

In an embodiment, a treatment may be applied to the body fluid. In an embodiment, a treatment may comprise exposing the body fluid to a binding antibody, for example, with the infusion. The binding be to an antigen specific to the spike protein of SARS-CoV-2. The specific antigen may include at least one antigen. The antigen may include the of SARS-CoV-2 spike glycoprotein. Other antigens may be included for testing as well. Other antigens may include Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex forming virion antibody complexes, and combinations thereof. The binding antibody and Covid-19 specific antigen form a virion antibody complex (FIL T Ab-TPA complex).

A neutralizing combination of two or more antibodies in an intravenous infusion against SARS-CoV2 spike protein, may be an effective treatment for Covid-19, especially in the early clinical stages of the illness. In an embodiment, two humanized neutralizing antibodies against the spike protein of SARS-CoV-2 may be used. These monoclonal antibodies may interact with the receptor binding domain (RBD) of SARS-CoV-2 and thereby may prevent its ability to infect cells. This may occur with picomolar binding affinity.

In an embodiment, the monoclonal antibody infusion may have the greatest utility in preventing hospitalization in outpatients early in their disease with Covid-19 infection, for example, within ten days of the onset of symptoms. The intravenous infusion may have great utility in treating patients 65 and older, those who have a BMI (body mass index) of 35 or greater, and those with other health conditions like diabetes, cardiovascular disease or chronic kidney disease.

In an embodiment, a total 2800 mg dose level will be utilized for the two monoclonal antibodies #1 and #2, which bind to distinct regions of the receptor binding domain of the spike protein of SARS-CoV-2. Other dosages may be used dependent upon severity of disease, strain, patient parameters, or the like. In infected patients this may give a greatly improved viral clearance around the third day after treatment with the intravenous infusion. The treatment mechanism of the intravenous antibody infusion may be due to humanized monoclonal antibodies binding directly with the spike protein of SARS-CoV-2, which prevents their ability to function physiologically. This process would also stimulate the removal of the antibody-virion complex by macrophages.

At 104, in an embodiment, the method may monitor the presence of the virion antibody complex. The monitoring may be from a body fluid sample, such as blood. The techniques described herein may be used to monitor the virion antibody complex.

Additionally or alternatively, a characteristic of the patient may be monitored. A characteristic may be blood urea nitrogen, serum creatinine, urine output, local infusion site reactions, headache, diarrhea, fatigue, back pain, nausea, pain in extremity, cough, upper respiratory tract infection, rash, pruritus, vomiting, abdominal pain, migraine, arthralgia, or the like. A characteristic may be any observation or measurement of the patient during the treatment period. For example, risk factors which may be involved in the utilization of the intravenous monoclonal antibody infusion would be hypercoagulable conditions, advanced age, a history of venous or arterial thrombosis, use of estrogens, hyperviscosity, and cardiovascular risk factors. For example, more common adverse reactions which may be expected to be observed in treatment subjects would be local infusion site reactions, headache, diarrhea, fatigue, back pain, nausea, pain in extremity, cough, upper respiratory tract infection, rash, pruritus, vomiting, abdominal pain, migraine, and arthralgia. However, these are commonly expected and treated clinical symptoms handled by health professionals in intravenous infusions.

Patients receiving the infusion may be monitored for renal function, including blood urea nitrogen, serum creatinine, and urine output in patients at risk of acute renal failure. Patents should be monitored for clinical signs and symptoms of hemolysis.

In an embodiment, patients may also be monitored for the virion antibody complex and/or ration or virus to antibody. For example, a body fluid such as blood may be drawn from a patient during treatment and monitored as a patient characteristic. The spike protein of SARS-CoV-2 antibody may contain an albumin moiety. This antibody may target and rapidly identify COVID-19 antigens. The antibody may or may not include a fluorescent tag. The fluorescent tag may be used for detection techniques. The fluorescent tag may be Alexa-488, Indocyanine green (ICG) or the like.

In an embodiment, the method may determine the presence or absence of the virion binding antibody complex. The binding antibody may include, for example, a fluorescent antibody, a luminous antibody, combinations thereof, or the like. The concentration of binding antibody may be made as high as necessary for the identification of extremely small, e.g., picogram/microliter, concentrations of the final virion binding antibody complex. The signal from the virion binding antibody complex may be amplified. The amplification may be necessary to identify a signal.

The method may utilize different techniques for determining the presence or absence of the virion binding antibody complex. As an example, a dialysis or a variant of dialysis may be used. The dialysis may be used to remove the fluorescent antibody-antigen complex. This may allow for a rapid identification of a COVID-19 sample. Such technique may be automated, controlled by a computer system, or the like. The system may use a threshold, limits, alarms, or the like.

In an embodiment, the methods may use flow cytometry analysis of fluorescent labelled antibodies relating to COVID-19. For example, flow cytometry analysis of fluorescent mAbs against SARS-CoV-2 spike (S) protein may be performed. For example, K562 cells may be fixed with 4% PFA (Paraformaldehyde) then permeabilized with 0.1% saponin in PBS (Phosphate-buffered saline). Cells may then be stained with anti-S mAb 1 or with mAb 1 that has been fluorescently labeled with Alexa488. Stained cells may be processed by flow cytometry.

In an embodiment, a method may utilize a designer fluorescent antibody with an attached macromolecular moiety. The macromolecular moiety, attached to the antibody, may be 1.000 mm to 0.00001 mm in diameter. Disclosed diameters are illustrative and may vary. The antibody-macromolecular moiety-targeted antigen complex would then be blocked for analysis, by using a series of microscreens which contain openings with a diameter 50.00000% to 99.99999% less than the diameter of the designer antibody-macromolecular moiety.

In an embodiment, methodology comprising the removal of the targeted antigen(s)/TA(s) by using a designer fluorescent antibody containing an iron (Fe) moiety. This will then create an Fe-fluorescent Antibody-Antigen (COVID-19/virion) complex. This iron containing complex may then be efficaciously removed using a strong, localized magnetic force field, which may be identified as positive.

In an embodiment, a variant of gel filtration chromatography, which may be utilized for the rapid identification of COVID-19. The fluorescent antibody-target antigen would be used to transport the sample through a size exclusion column that would be used to separate the fluorescent antibody-target antigen by size and molecular weight.

In an embodiment, a methodology using a molecular weight cutoff filtration may be employed. Molecular weight cut-off filtration refers to the molecular weight at which at least or approximately 80% of the target antigen(s)/TA(s) may be prohibited from membrane diffusion.

In an embodiment, a removal methodology for the fluorescent antibody-target antigen(s) may be used. The removal methodology may be selected from a group comprising a mechanical filter, a chemical filter, a dialysis machine, a molecular filter, molecular adsorbent recirculating system (MARS), a plasmapheresis unit, or combinations thereof.

For example, virions may be captured using antibody microarrays. The microarray may contain one or more binding antibody. In an embodiment, the binding antibody may comprise a fluorescent antibody (FI). In another embodiment, the binding antibody may comprise a luminescent (Lu) antibody. The microarray may comprise a plurality of antibodies fixed on a solid surface. The solid surface may be any suitable material. The microarray material may be transparent, such as glass, plastic, silicon, combinations thereof, or the like. The microarray may allow detection of at least one virion antibody complex. The microarray can comprise a plurality of monoclonal antibodies attached at high density on the solid surface. Typically, the microarray may contain millions of antibodies. Exposure of the virion to the binding antibodies on the microarray creates the virion. The complex may be tracked using an appropriate sensor. To identify the virion antibody complex after exposure in the microarrays, the body fluid may then be forced through a container preferably constructed from a transparent material, which exposes the virion antibody complex to a light-sensing device. The sensing device may also create an enlarged, magnified visual image of virion antibody complex. A concentrated and focused intense energy beam, such as light, is then used to properly illuminate the virion antibody complex within the body fluid. Each virion antibody complex may be rapidly identified. The virion antibody complex may also be identified and tracked using optical or digital enhancement or magnification.

At 105, in an embodiment, if a virion complex cannot be determined or is outside acceptable levels, the method may continue to treat the patient. For example, virus levels remain high within the patients and infusion is continued. Alternatively, the method may determine that the patient body fluid does not contain the virion. For example, the patient does not have COVID-19 or virus levels are within acceptable levels. Additionally or alternatively, the system may output an alarm, log an event, or the like. If a characteristic or antibody virion complex can be determined, the system may provide an output at 106. For example, the virion binding antibody complex determination or another measure of the patient's status may be an output that is provided to a device in the form of a display, printing, storage, audio, haptic feedback, or the like. Alternatively, or additionally, the output may be sent to another device through wired, wireless, fiber optic, Bluetooth®, near field communication, or the like.

The various embodiments described herein thus represent a technical improvement to a treatment for COVID-19 or mutations thereof using antibodies directed to the spike protein of SARS-CoV-2. Using the techniques as described herein, an embodiment may use a method or treatment to reduce and/or eliminate the ability of the SARS-CoV-2 virus to infect further cells in a patient.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the FIGURES, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for treatment of the COVID-19 virus, comprising:
preparing an intravenous infusion, wherein the intravenous infusion comprises at least one humanized monoclonal antibody against the SARS-CoV-2 spike protein;
introducing the intravenous infusion to a patient;
forming a virion antibody complex, wherein the virion antibody comprises the at least one humanized monoclonal antibody against the SARS-CoV-2 spike protein; and
monitoring the virion antibody complex from a sample of a body fluid.

2. The method of claim 1, wherein the at least one humanized monoclonal comprises a fluorescent tag.

3. The method of claim 2, wherein the fluorescent tag is selected from the group consisting of: Alexa-488 and Indocyanine green.

4. The method of claim 1, wherein the at least one humanized monoclonal antibody comprises an iron moiety, wherein a localized magnetic field removes the virion antibody complex from the body fluid.

5. The method of claim 1, further comprising removing the presence of the virion binding antibody complex using a technique selected from the group consisting of: a flow cytometry analysis, a macromolecular entity, gel filtration chromatography, a molecular weight cutoff filtration, and a microarray.

6. The method of claim 1, further comprising at least one additional monoclonal antibody from the group consisting of: Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

7. The method of claim 1, further comprising monitoring a characteristic of the patient.

8. The method of claim 7, wherein the characteristic comprises at least one renal function.

9. The method of claim 1, further comprising dialysis of the virion antibody complex from the body fluid.

10. The method of claim 1, wherein the treatment is performed at the early clinical stages of the onset of COVID-19.

11. A treatment kit for treatment of the COVID-19 virus, comprising:
prepare an intravenous infusion, wherein the intravenous infusion comprises at least one humanized monoclonal antibody against the SARS-CoV-2 spike protein;
introduce the intravenous infusion to a patient;
form a virion antibody complex, wherein the virion antibody comprises the at least one humanized monoclonal antibody against the SARS-CoV-2 spike protein; and
monitor the virion antibody complex from a sample of the body fluid.

12. The kit of claim 11, wherein the at least one humanized monoclonal comprises a fluorescent tag.

13. The kit of claim 12, wherein the fluorescent tag is selected from the group consisting of: Alexa-488 and Indocyanine green.

14. The kit of claim 11, wherein the at least one humanized monoclonal antibody comprises an iron moiety, wherein a localized magnetic field removes the virion antibody complex from the body fluid.

15. The kit of claim 11, further comprising removing the presence of the virion binding antibody complex using a technique selected from the group consisting of: a flow cytometry analysis, a macromolecular entity, gel filtration chromatography, a molecular weight cutoff filtration, and a microarray.

16. The kit of claim 11, further comprising at least one additional monoclonal antibody from the group consisting of: Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

17. The kit of claim 11, further comprising monitoring a characteristic of the patient.

18. The kit of claim 17, wherein the characteristic comprises at least one renal function.

19. The kit of claim 11, further comprising dialysis of the virion antibody complex from a body fluid.

20. A method for treatment of the COVID-19 virus, comprising:
preparing an intravenous infusion, wherein the intravenous infusion comprises at least one humanized monoclonal antibody against the SARS-CoV-2 spike protein;
introducing the intravenous infusion to a patient;
forming a virion antibody complex, wherein the virion antibody comprises the at least one humanized monoclonal antibody against the SARS-CoV-2 spike protein;
monitoring the virion antibody complex from a sample of a body fluid; and
removing the virion antibody complex from the body fluid.
